(19) United States
(12) Patent Application Publication
(10) Pub. No.: US 2014/0331863 A1
(43) Pub. Date: Nov. 13, 2014

(54) Integrated Canister Shut-Off Valve and Filtration System

(71) Applicant: Essentra Porous Technologies Corp., Colonial Heighs, VA (US)

(72) Inventors: Jian Xiang, Midlothian, VA (US); Geoffrey M. Stoltz, Moseley, VA (US)

(73) Assignee: Essentra Porous Technologies Corp., Colonial Heights, VA (US)

(21) Appl. No.: 14/272,923

(22) Filed: May 8, 2014

Related U.S. Application Data
(60) Provisional application No. 61/820,898, filed on May 8, 2013.

(51) Int. Cl. A61M 1/00

(52) U.S. Cl. CPC A61M 1/0005
USPC 96/134

(57) ABSTRACT

A shut-off valve is provided for use in a suction canister. The shut-off valve comprises a valve portion comprising a valve body having at least one side wall and an end wall collectively defining a valve interior. The valve body has an open end configured to allow fluid communication between the valve interior and an outlet port of the canister. The valve body walls comprise a porous plastic material and a moisture-reactive material adapted to expand on contact with liquid and reduce or eliminate the flow path through the porous plastic material. The shut-off valve further comprises a filter portion covering at least a portion of an exterior surface of the valve body. The filter portion comprises a fiber filter medium comprising a plurality of fibers collectively defining a tortuous fluid flow path through the fiber filter medium.

### CROSS-REFERENCE TO RELATED APPLICATIONS

This application claims priority to U.S. Provisional Application No. 61/820,898, filed May 8, 2013, which is incorporated herein by reference in its entirety.

### BACKGROUND OF THE INVENTION

The present invention relates generally to the field of filtration devices and, more particularly, to aerosol/liquid filters comprising three dimensional sintered beads and bonded fiber structures.

During surgical procedures, vacuum systems are widely used to remove body fluids, aerosols, and debris from the surgical area. These materials are biohazards and are typically contained in a device widely known in the field as a surgical suction canister. With reference to FIGS. 1A and 1B, a typical surgical suction canister 10 has a container body 12 with a cover assembly 14 having two opening ports 16, 18. One port (outlet port) 18 is connected to a vacuum pump or similar device (not shown) to provide a partial vacuum in the interior 13 of the canister body 12. The other port (inlet port) 16 is provided to draw body fluids, aerosols and other surgical debris into the canister interior 13 during the operation. The inlet port 16 may be or comprise multiple ports connected to multiple tubes from the patient. Some systems also contain a larger diameter dump port to empty the canister 10.

It is imperative that equipment downstream of the outlet port not be contaminated by biohazardous surgical debris. To assure this, the surgical suction canister 10 may be fitted with a protective shut-off valve 50 upstream of the outlet port 18. The purpose of the shut-off valve 50 is to prevent biohazardous debris from contaminating equipment or spaces downstream of the outlet part 18. Early versions of these shut-off valves were typically mechanical in design, utilizing a “floating ball” system, which will shut off the outlet port when liquid levels reach the height of the valve 50 as shown in FIG. 1B. More recently, surgical canisters have been fitted with a filtration/self-sealing system comprising a porous plastic filter structure formed from sintered plastic beads. Filters of this type may be imbibed with a moisture-reactive powder, such as polysaccharide, polyacrylate or certain proteins, which serves to block the filter when challenged with aqueous liquids or aerosols, thus preventing potential contamination of equipment or spaces downstream of the filter. Such moisture-reactive agents are dormant until the filter/valve 50 is contacted by aqueous liquid or aerosol. As soon as the liquid starts to penetrate into the filter/valve 50, the liquid causes the powder to swell and form a colloidal gel. This cohesive gel structure serves to shut off the flow of fluids through the valve 50 and outlet port 18, thus protecting articles downstream.

A key issue with porous plastic filters is the wide use of hot, cauterizing knives and lasers in modern surgery. These devices generate smoke which has been observed to plug, or “blind off”, these porous plastic filters, resulting in their premature blocking. In many cases, the filters blind off before the liquid level inside the canister reaches the valve. In other cases, the air flow through the filter is so impeded that the effectiveness of the suction devices used in surgery is compromised. This creates a problem in the surgical theater in that medical personnel are often called upon during a surgical procedure to either change the lid containing the valve, or change out the entire canister system. While not only distracting from the surgical procedure itself, such change-outs have the dangerous potential of introducing biohazardous materials into the surgical theater.

To combat this issue, some manufacturers have installed an additional filter upstream of the shutoff valve. This filter is configured to remove smoke and steam aerosols from the fluid before they reach the shutoff valve, thereby improving the valve's longevity (i.e., the length of time it will operate without blinding off). Typically, the additional filter is a non-woven planar sheet formed from material such as fiberglass and disposed in a housing that can be secured to or otherwise positioned upstream of the shutoff valve. While these filters have, in certain cases, been effective at extending the life of the shutoff valve, they add to the cost and complexity of the system and result in the need to replace two discreet filters instead of one.

### SUMMARY OF THE INVENTION

An illustrative aspect of the invention provides a shut-off valve for use in a suction canister having an outlet port through which a suction force is applied to an interior of the canister, the shut-off valve comprising a valve portion configured for attachment to the suction canister interior at the outlet port. The valve portion comprises a valve body having at least one side wall and an end wall collectively defining a valve interior. The valve body has an open end generally opposite the end wall. The open end is configured to allow fluid communication between the valve interior and the outlet port. The valve body walls comprise a porous plastic material configured to provide a flow path between the interior of the canister and the interior of the valve body and a moisture-reactive material adapted to expand on contact with and absorption of a liquid. The expanded moisture-reactive material acts to reduce or eliminate the flow path through the porous plastic material. The shut-off valve further comprises a filter portion covering at least a portion of an exterior surface of the valve body. The filter portion comprises a fiber filter medium comprising a plurality of fibers collectively defining a tortuous fluid flow path through the fiber filter medium, the filter portion being configured and positioned so that at least a portion of a fluid drawn into the valve interior passes through the fiber filter medium before passing through the valve body into the valve interior and, thence, to the outlet port.

### BRIEF DESCRIPTION OF THE DRAWINGS

The invention can be more fully understood by reading the following detailed description together with the accompanying drawings, in which like reference indicators are used to designate like elements, and in which:

FIG. 1A is a perspective view of a surgical suction canister with a quantity of fluid disposed therein;

FIG. 1B is a perspective view of the surgical suction canister with a greater quantity of fluid disposed therein;

FIG. 2 is a top view of a shut-off valve according to an embodiment of the invention;

FIG. 3 is a section view of the shut-off valve of FIG. 2;

FIG. 4 is a perspective view of a shut-off valve according to an embodiment of the invention;

FIG. 5 is a graphical representation of flow rate test data for shut-off valves according to embodiments of the invention;

FIG. 6 is a graphical representation of longevity performance data for shut-off valves according to embodiments of the invention; and

FIG. 7 is a graphical representation of longevity performance data for shut-off valves according to embodiments of the invention.

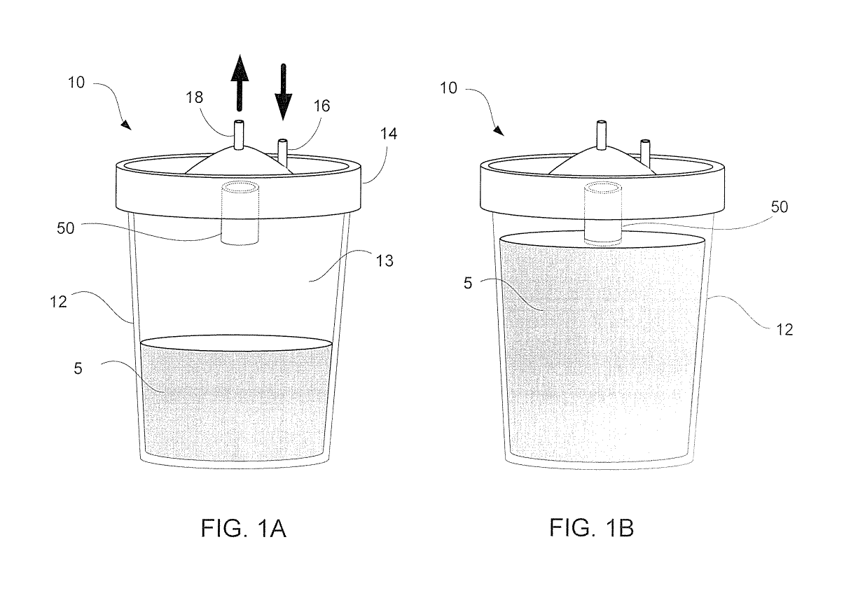

### DETAILED DESCRIPTION OF THE INVENTION

While the invention will be described in connection with particular embodiments, it will be understood that the invention is not limited to these embodiments. On the contrary, it is contemplated that various alternatives, modifications and equivalents are included within the spirit and scope of the invention as described

Various embodiments of the invention provide an integrated filter/valve device having a shutoff valve portion configured for closing the exit port of a suction canister when the liquid in the canister reaches the level of the device and a prefilter portion configured to preclude particulate and aerosol matter from reaching the shutoff portion.

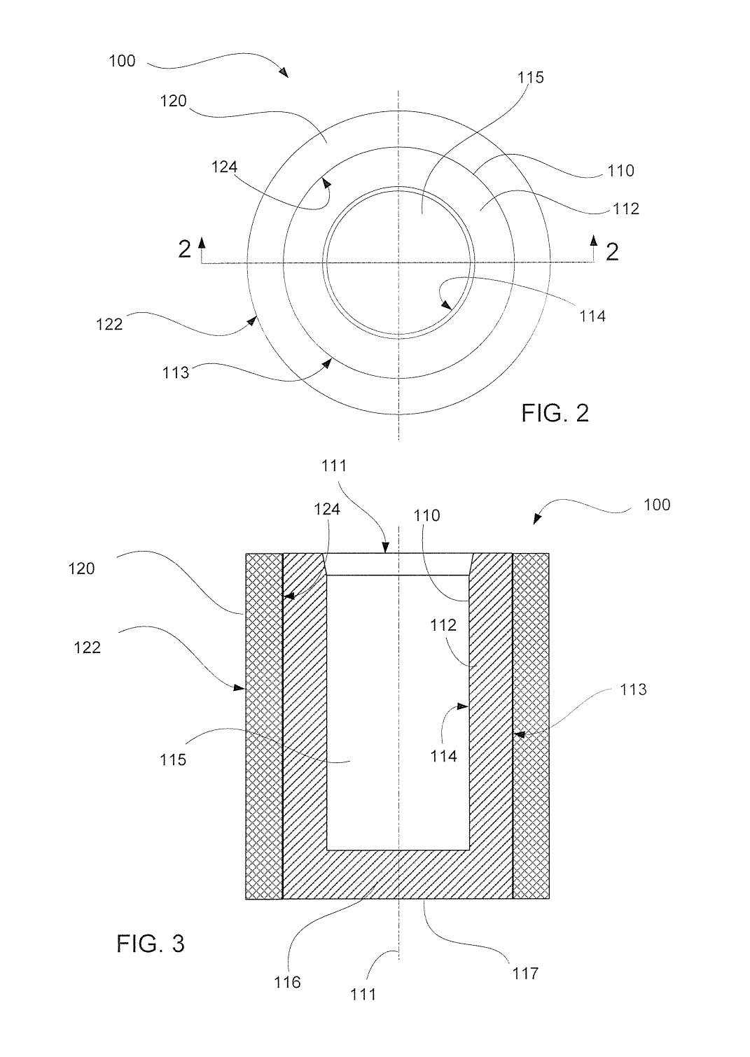

With reference to FIGS. 2-4, a canister shutoff valve 100 according to an embodiment of the invention comprises an inner filter structure 110 that acts as a self-sealing valve portion and an outer filter structure 120 having a fiber filter medium that acts as a pre-filter to screen particulate and aerosol matter from the inner filter portion 110. In the illustrated embodiment, the inner filter structure 110 is an annular cylinder open at one end 111. The inner filter structure 110 has a cylindrical wall 112 having an outer surface 113 and an inner surface 114 and has a base wall 116 at its closed end. The cylindrical wall 112 and the base wall 116 combine to define a valve interior space 115. The base wall has an outer surface 117.

Some or all of the inner filter structure 110 may be formed as a porous filter and in particular embodiments is or comprises a porous plastic filter (PPF). The PPF may be formed from sintered ultrahigh molecular weight polyethylene (UHMWPE) or other suitable sinterable materials such as polypropylene, polystyrene, polytetrafluoroethylene, and other high viscosity thermoplastic polymer beads and powders.

The PPF may further comprise a moisture-reactive material adapted to react and block the passages of the PPF when challenged with aqueous liquids or aerosols. This material may be or comprise polyacrylate or carboxymethyl cellulose (CMC) or other suitable material.

In some embodiments, the PPF may be formed as a composite material comprising a blended combination of the porous plastic material and the moisture-reactive material. For example, the PPF could be formed by sintering or molding a powder mixture of dry resin and flow barrier material. In the resulting material, the plastic particles are aggregated to form a porous material with tortuous passageways throughout. The moisture-reactive material is disposed uniformly through the composite material. The composite material is configured to allow passage of gaseous fluids. The composite material encounters a liquid, however, the liquid interacts with the moisture-reactive material, which expands to close off the pathways through the composite material.

In other embodiments, the PPF may be formed so that the plastic material is pre-formed separately from the moisture-reactive material, which is later disposed within or at the surface of the formed porous plastic material.

While the inner filter 110 illustrated in FIGS. 2-4 is cylindrical, it will be understood that a PPF may be formed with any desired cross-section and wall thickness. It may, for example have any axisymmetric shape with the cross-section varying along the axis 111. Thus, PPF may be formed with a slight taper so as to make it frusto-conical rather than cylindrical. Alternatively, the PPF may be formed as a combination of cylinders having different diameters aligned in tandem along the axis 111. In other embodiments, the inner filter 110 may have a polygonal or other non-axisymmetric cross-section.

In any of the above embodiments, the wall thickness and porosity of the inner filter 110 may be selected based on desired flow and filtration properties.

The outer filter structure or pre-filter 120 of the integrated shutoff valve 100 is a cylindrical sleeve having an outer surface 122 and an inner surface 124. The outer filter structure 120 is coaxially positioned to surround the inner filter structure 110 with the inner surface 124 in intimate contact with the outer surface 113 of the inner filter structure 110.

In some embodiments, the outer filter structure 120 comprises a plurality of fibers bonded to one another at spaced apart points of contact to form a porous, three-dimensional, self-sustaining, bonded fiber structure. In other embodiments, the outer filter structure may be formed from a plurality of tightly bundled but unbonded fibers surrounded by one or more permeable retaining or support layers. The permeable layer may be a membrane, sheath, or woven or non-woven fiber layer.

The outer filter structure 120 may be formed as a separate tube-like structure having an inside diameter that is slightly smaller than the outside diameter of the inner filter structure 110. The outer filter structure 120 may then by pressed over the inner filter structure 110 with the resultant friction or interference fit producing intimate contact and an interface between the two structures. In alternative embodiments, the outer filter structure 120 may be formed directly over the inner filter structure 110. In some embodiments, the outer filter structure 120 may be formed from a planar member formed into a tube or wrapped directly around the outer circumference of the inner filter structure 110.

Like the valve portion 110 of the integrated shutoff valve 100, the outer filter portion 120 need not be formed as a cylinder. Indeed, the outer filter portion may be formed in any shape necessary to conform to the cross-sectional shape of the valve portion 110. As is discussed below, bonded fiber structures may be readily formed in any cross-sectional configuration, including axisymmetric and non-axisymmetric cross-sections.

In some embodiments, the outer filter portion 120 may be configured to overlie only a portion of the inner filter structure. As is shown in FIGS. 2-4, the outer filter portion 120 may be configured to overlie all of the exposed surface of the inner filter structure 110 except the base wall surface 117. Alternatively or in addition, the outer filter portion may be configured to overlie only a portion of the cylindrical wall surface 113. The remainder of the cylindrical wall surface 113 may be exposed to the interior of the canister or closed off by a separate cover or portion of the canister.

As noted above, the outer filter structure 120 may be formed as a bonded fiber structure. In general, bonded fiber components and structures are formed from webs of thermoplastic fibrous material comprising an interconnecting network of highly dispersed continuous or staple fibers bonded to each other at points of contact. These webs can be formed into substantially self-sustaining, three-dimensional porous components having high surface areas and porosity, and may be formed in a variety of sizes and shapes.

The bonded fiber structure of the outer filter structure 120 may be formed from a plurality of fibers comprising either bicomponent fibers, monocomponent fibers, or both. The term “bicomponent fiber” as used herein refers to the use of two polymers of different chemical nature placed in discrete portions of a fiber structure. While other forms of bicomponent fibers are possible, the more common techniques produce either “side-by-side” or “sheath-core” relationships between the two polymers.

In an exemplary embodiment, inner fiber portion 120 of an integrated shut-off valve 100 may be formed from or include sheath-core bicomponent fibers where the sheath is polyethylene terephthalate (PET) and the core is polypropylene (PP), as is disclosed in U.S. Pat. Nos. 5,607,766 and 5,620,641. Such bicomponent fibers may be formed into a self-sustaining cylinder with high dimensional tolerance that can be applied over top of the inner filter structure.

In some embodiments, the fibers of the outer filter portion 120 may comprise sheath-core bicomponent fibers in which the sheath polymer is polyethylene or copolymers of polyethylene and the core is polypropylene. In other embodiments, the fibers may comprise sheath-core bicomponent fibers where the sheath polymer is PET and the core polymer is polybutylene terephthalate (PBT).

In some embodiments, the fibers of the outer filter structure 120 may comprise or consist entirely of monocomponent fibers. In particular embodiments, the outer filter structure may comprise a blend of bicomponent and monocomponent fibers or multiple different bicomponent fiber types as described in U.S. Pat. Nos. 6,103,181, 6,330,833, 6,576,034, 6,596,049, 6,602,311, and 6,616,723, which are incorporated herein by reference in their entireties. As disclosed in these references, bonded fiber structures may be formed from a homogeneous or uniform mixture of monocomponent and multiple-component fibers, or even a uniform mixture of different multiple-component fibers.

As used herein to describe the bonded fiber structures of the invention, “self-sustaining” means that the bonded fiber structure is not dependent on another structure (e.g., a sheath or cover) to maintain its structural form and integrity and its flow properties. Examples of such structures and methods for making them may be found in U.S. Pat. Nos. 5,607,766; 5,620,641; 5,633,082; 6,460,985; 6,840,692; 7,290,668; and 7,888,275 and European Patent Pub. Nos. EP0881889 and EP1230863, the complete disclosures of which are incorporated herein by reference in their entireties.

The polymeric fibers themselves may be produced by a number of common techniques, oftentimes dictated by the nature of the polymer and/or the desired properties and applications for the resultant fibers. Among such techniques are conventional melt spinning processes, wherein a molten polymer is pumped under pressure to a spinning head and extruded from spinerette orifices into a multiplicity of continuous fibers. Melt spinning techniques are commonly employed to make both mono-component and bi or multi-component fibers. In addition, some polymers can be dissolved in a suitable solvent (e.g., cellulose acetate in acetone) of typically 25% polymer and 75% solvent. In a wet spinning process, the solution is pumped at room temperature through the spinerette which is submerged in a bath of a liquid non-solvent in which the non-solvent serves to coagulate the polymer to form polymeric fibers. It is also possible to dry spin the fibers into hot air (or other hot gas), rather than a liquid bath, to evaporate the solvent and form a solid fiber strand. These and other common spinning techniques are well known in the art.

After spinning, the fibers are typically attenuated. Attenuation can occur by drawing the fibers from the spinning device at a speed faster than their extrusion speed, thereby producing fibers which are finer, i.e. smaller in diameter. This attenuation may be accomplished by taking the fibers up on rolls rotating at a speed faster than the rate of extrusion. Attenuation may also be accomplished by drawing the fibers utilizing draw rolls operating at different speeds. Depending on the nature of the polymer, drawing the fibers in this manner may orient the polymer chains, thus improving the physical properties of the fiber. Melt-spinning, as described above and as known in the art, is a typical method of making both mono-component and bicomponent fibers.

Mono-component, bicomponent, and multi-component fibers may be formed by melt blowing. Briefly, melt-blowing involves the use of a high speed, typically high temperature gas stream at the exit of a fiber extrusion die to attenuate or draw out the fibers while they are in their molten state. See, for example, U.S. Pat. Nos. 3,595,245, 3,615,995 and 3,972,759 the complete disclosures of which are incorporated herein in their entirety by reference, for a comprehensive discussion of the melt blowing processing. The fine fibers are commonly collected as an entangled web on a continuously moving surface, such as a conveyor belt or a drum surface, for subsequent processing.

Depending on the nature of the fibers, they may be formed into tows, loosely bonded into a web or otherwise gathered together and are typically passed through one or more processing stations in which the fibers are bonded and formed to produce a continuous, self-sustaining, porous structure. The bonding process may involve drawing the fibers a heated die in which the temperature is at or near the melt temperature of at least one of the fiber materials. As the fibers are heated, the die force them into contact with one another at various spaced-apart points along their lengths. At those points where contact is made with the melted fiber component material, a bond is formed that is fixed and retained upon cooling. Thus, the fibers remain bonded at these contact points, thereby producing a self-sustaining fiber structure.

In certain embodiments, bonded fiber structures may be formed by directly depositing newly spun fibers on a body such as a mandrel or a core material intended to be retained in a final product. In some instances, a bonded fiber structure may be formed as an axisymmetric body by directly depositing fibers on a rotating axisymmetric body.

The final product of the methods described above is a self-sustaining network of bonded bicomponent fibers. This network defines a tortuous flow path for passage of fluids through the wick and provides for interstitial entrapment of loaded substances and/or substances entrained in fluids passing therethrough.

The fibers used in the various embodiments of the invention may have any diameter suitable for providing desired flow and filtration characteristics. In some embodiments, some or all of the fibers may have a diameter in a range of 1 micron to 100 microns. Such fibers are referred to herein as microfibers.

In some embodiments, a bonded fiber structure may be used to form an outer filter portion that comprises or consists entirely of melt blown nanofibers (i.e., fibers having a diameter in a range of 0.1 micron to 1 micron). These fibers may be either monocomponent or bicomponent fibers formed from polypropylene, polyethylene, PET or other polyesters, Nylon-6 or other polyamides, and/or other thermoplastic polymers.

A bonded fiber structure used to form the outer filter portion of the integrated shut-off valve of the invention may be substantially homogeneous through its thickness or may be selectively variable to provide a depth filter. This may be accomplished by varying the fiber material, type, or diameter through the thickness of the structure wall. In some embodiments, the wall of the outer filter portion may be formed from multiple fiber structure layers, each layer having its own material and flow properties. For example, a bonded fiber outer filter structure may be formed with one or more microfiber layers in combination with a layer comprising or consisting of nanofibers. Such structures are described in detail in U.S. patent application Ser. No. 12/706,729, filed Feb. 17, 2010, the full disclosure of which is incorporated herein by reference in its entirety. The layers of such structures are preferably integrally formed as a single bonded fiber structure. Alternatively, separately formed bonded fiber structures may be bonded together to form a single layered structure.

While the fibers of the bonded structures used in the invention are typically bonded by thermal means, it will be understood that they may also be bonded by chemical or mechanical means.

As noted above, some embodiments may use tightly bundled but unbonded fibers to form the tortuous passages required for the outer filter portion. Such fibers may include fibers formed from any of the previously discussed materials. They may also include glass fibers. Any of these fibers may be formed as either microfibers or nanofibers. In such embodiments, the outer filter portion 120 may be formed as a layer of bundled fibers supported by a retaining layer on one or both sides. In an illustrative embodiment, a layer of bundled, unbonded fibers may be held in close contact with at least a portion of the outer surface of the inner filter portion 110 by a permeable retaining layer. This retaining layer serves to maintain the relative spatial relationships of the fibers to one another and the tortuous passages there through the fibers. The permeable retaining layer also provides a passable boundary between the canister interior and the bundled fibers. In this embodiment, a fluid must pass through the outer permeable retaining layer the bundled, unbonded fiber layer before passing through the underlying portion of the inner filter portion 110. In some embodiments, the bundled fiber layer may have a second permeable retaining layer between the bundled fibers and the outer surface of the inner filter portion 110.

The permeable retaining layer may be a woven or non-woven fiber layer or may itself be a bonded fiber structure having a higher porosity than the bundled fiber layer. Alternatively, the permeable retaining layer may be any form of permeable membrane formed from a material compatible with the fluids involved in the application and having sufficient strength to retain the bundled fibers.

In a particular embodiment, the bundled fiber layer may comprise a plurality of glass fibers held between inner and outer fibrous retaining layers. The glass fibers may be or comprise nanofibers and the retaining layers may comprise a plurality of woven or nonwoven polymeric fibers.

The integrated shutoff valve 100 combines the self-sealing features of a PPF shutoff valve with the particle/aerosol filtration features of a three-dimensional, bonded fiber structure. In use, the integrated valve may be disposed at the exit port or suspended from the exit port within the canister interior. In either case, suctioned gas brought into the canister by the vacuum suction, which may contain liquid or solid biological material and/or other particulate matter, is drawn through the outer filter structure and through the walls of the inner filter structure. The bonded fiber structure removes smoke and other particles before they reach the inner filter element, thereby extending the longevity of the inner element. The outer filter structure may also be used to filter out liquid aerosol particles that would otherwise penetrate the walls of the inner filter portion and interact with the moisture-reactive material, thereby causing the restriction or closure of flow passageways through the inner filter portion.

In the illustrated embodiment, the base wall 116 is left uncovered. If the base wall is formed from the same porous material as the cylindrical wall 112, suctioned fluid will pass through the outer surface 117 of the base wall and through into the interior 115 of the inner filter element. In some embodiments, however, the base wall may be formed by or covered by a non-porous material. Alternatively, an outer wall of filter material may be applied over the base wall 116. Such a wall may be formed as part of the outer filter structure 110 or may be formed and applied separately. In the latter case, the material of this base cover wall may be the same as or different from the material of the outer filter structure.

It will be understood that the shutoff valve 100 can be sized for any particular application or for incorporation into any canister system. In typical surgical applications, the inner filter structure (i.e., the PPF in certain embodiments) may have an outside diameter (OD_{PPF}) in a range from 10 mm to 25 mm and a wall thickness in a range of 2 mm to 5 mm. In particular embodiments, the wall thickness is in a range of 3 mm to 4 mm and may have a nominal thickness of 3.5 mm. In such typical applications, the outside filter structure may have an outside diameter (OD_{PF}) in a range of about 12 mm to 35 mm and a wall thickness in a range of 2 mm to 5 mm. In particular embodiments, the wall thickness is in a range of 3 mm to 4 mm and may have a nominal thickness of 3.5 mm. The length or height H of the valve body is virtually unlimited. In typical applications, however, the length H will be in a range of 25 mm to 75 mm.

### EXAMPLES AND TESTING

A number of integral filter/valve devices were constructed and tested to determine the efficacy of the integral filter in increasing the longevity of the inner filter. In the test devices, the inner filter structure was formed as a PPF comprising sintered UHMWPE beads containing carboxymethyl cellulose as a moisture blocking filler. The devices incorporated bonded fiber outer filter structures held to the PPF by a friction fit. The outer filter elements were formed from PET sheath/PP core or polyethylene sheath/PP core bicomponent fibers. Fiber size (cross sectional diameters) for the PET/PP bicomponent fibers was measured at 7, 10 and 14 microns for fiber densities of 0.06 to 0.10 g/cc. Fiber size for the PE/PP bicomponent fibers was measured at 20-50 microns.

Each device was tested by affixing the device to a vacuum pump, with a pump setting to draw approximately 30 liters of air per minute through a clean filter. To the inlet side of the filter was connected a fixture designed to hold a cigarette. Cigarette smoke was used as a model system for surgical cauterization smoke. Cigarettes were sequentially smoked by the machine with the flow drawn through the test device until the flow rate was decreased to 5 liters per minute, at which point the PPF was deemed to be plugged. For purposes of this study, the “longevity” of the device was deemed to be the number of smoked cigarettes needed to reduce the flow rate to 5 liters per minute.

PPFs without an outer filter structure were used as a control. These typically plugged after 6-8 cigarettes.

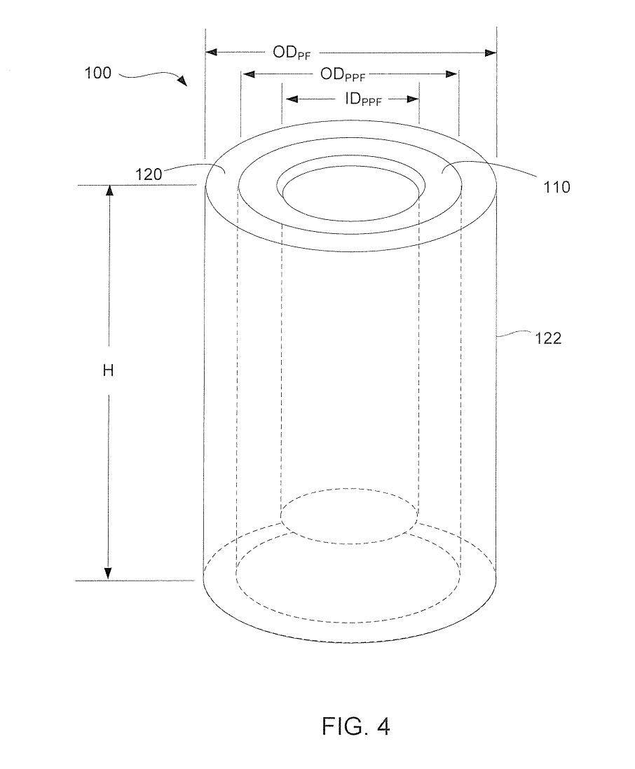

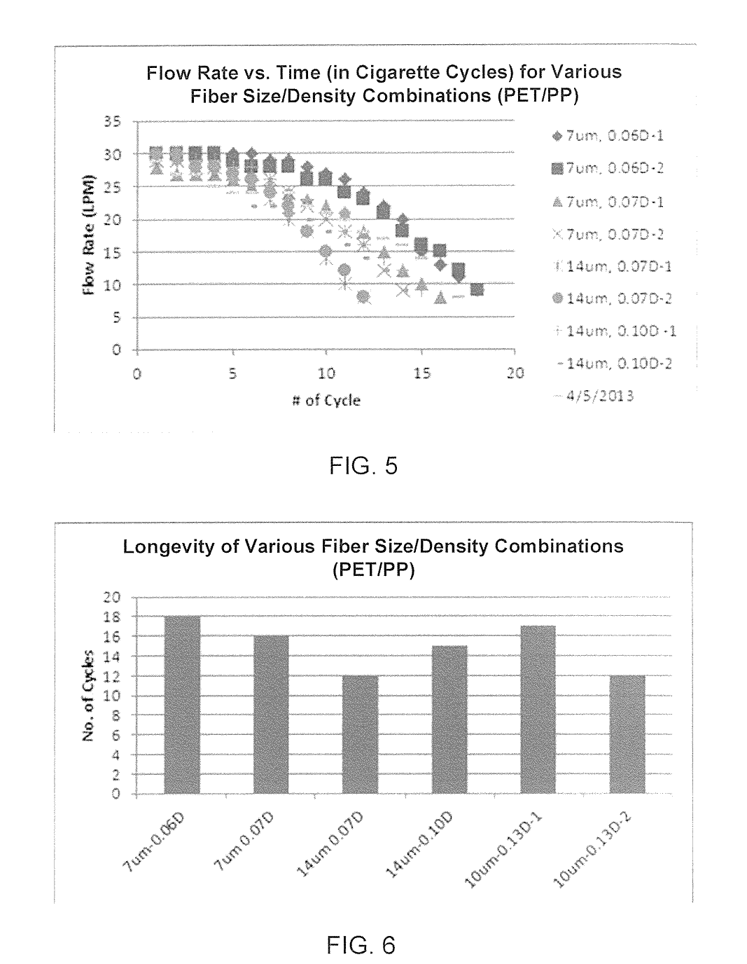

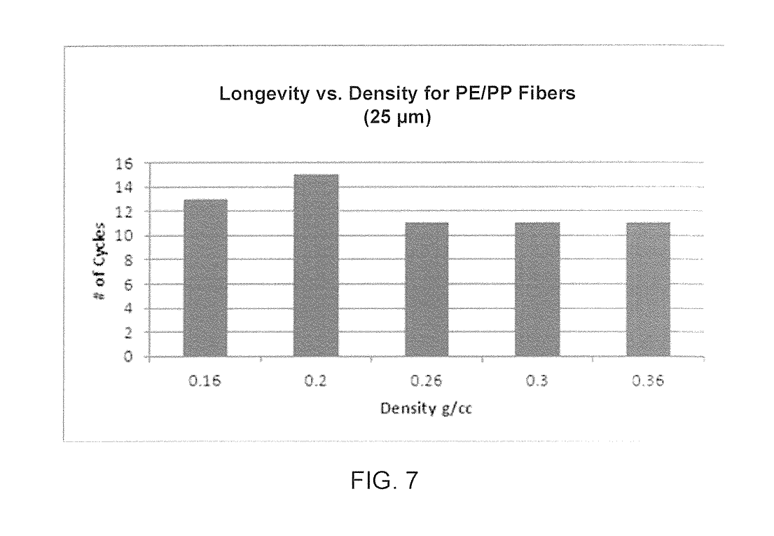

Test results for the PET/PP fiber outer filter are shown in FIGS. 4 and 5 in which a “cycle” means 1 smoked cigarette. As can be observed, the longevity of the filter unit is significantly increased by incorporation of the bonded fiber outer filter as compared to the control. Based on this data, efficiency appears to be the greatest at smaller fiber sizes and lower density.

Test results for the PE/PP fiber outer filter are shown in FIG. 6. In this case resistance to plugging is poorer than observed with the PET/PP filter, the difference being due to the larger fiber diameter.

In addition to the above data, a bonded fiber outer filter structure formed from PP nanofibers having diameters in the range of 0.5 to 1.0 micron produced a PPF longevity in excess of 50 cigarettes.

It will be readily understood by those persons skilled in the art that the present invention is susceptible to broad utility and application. Many embodiments and adaptations of the present invention other than those herein described, as well as many variations, modifications and equivalent arrangements, will be apparent from or reasonably suggested by the present invention and foregoing description thereof, without departing from the substance or scope of the invention.

What is claimed is:

1. A shut-off valve for use in a suction canister having an outlet port through which a suction force is applied to an interior of the canister, the shut-off valve comprising:
a valve portion configured for attachment to the suction canister interior at the outlet port, the valve portion comprising a valve body having at least one side wall and an end wall collectively defining a valve interior, and an open end generally opposite the end wall, the open end being configured to allow fluid communication between the valve interior and the outlet port, the valve body walls comprising
a porous plastic material configured to provide a flow path between the interior of the canister and the interior of the valve body and
a moisture-reactive material adapted to expand on contact with and absorption of a liquid, the expanded moisture-reactive material acting to reduce or eliminate the flow path through the porous plastic material; and
a filter portion covering at least a portion of an exterior surface of the valve body, the filter portion comprising a fiber filter medium comprising a plurality of fibers collectively defining a tortuous fluid flow path through the fiber filter medium, the filter portion being configured and positioned so that at least a portion of a fluid drawn into the valve interior passes through the fiber filter medium before passing through the valve body into the valve interior and, thence, to the outlet port.

2. A shut-off valve according to claim 1, wherein
at least a portion of the valve body is an annular cylinder having an outer cylindrical surface, and
the fiber filter medium is formed as an annular cylindrical sleeve having an exposed outer surface and an inner surface in contact with the outer cylindrical surface of the valve body.

3. A shut-off valve according to claim 1, wherein the plurality of fibers comprises nanofibers having diameters in a range of 0.1 micron to 1 micron.

4. A shut-off valve according to claim 3, wherein the nanofibers are glass fibers.

5. A shut-off valve according to claim 3, wherein the nanofibers are monocomponent polymer fibers comprising one of the set consisting of polypropylene (PP), polyethylene (PE), polyethylene terephthalate (PET), and Nylon-6.

6. A shut-off valve according to claim 3, wherein the plurality of fibers further comprises microfibers having diameters in a range of 1 micron to 100 microns.

7. A shut-off valve according to claim 1, wherein the plurality of fibers comprises sheath-core polymer bicomponent fibers formed with at least one of the set consisting of a PET sheath and PP core, a PE sheath and a PP core, a PE copolymer sheath and a PP core, a PET sheath and a polybutylene terephthalate (PBT) core.

8. A shut-off valve according to claim 1, wherein the filter portion has a thickness in a range of 2 mm to 5 mm.

9. A shut-off valve according to claim 1, wherein the filter portion has a thickness in a range of 3 mm to 4 mm.

10. A shut-off valve according to claim 1 wherein the filter medium is or includes a porous, self-sustaining, bonded fiber structure formed from the plurality of fibers, the plurality of fibers comprising polymeric fibers bonded to one another at spaced apart points of contact.

11. A shut-off valve according to claim 10, wherein the bonded fiber structure is formed from a plurality of bonded fiber layers, each layer being formed from polymeric fibers bonded to one another at spaced apart points of contact.

12. A shut-off valve according to claim 11, wherein at least one of the bonded fiber layers comprises nanofibers having diameters in a range of 0.1 micron to 1 micron.

13. A shut-off valve according to claim 1 wherein the plurality of fibers are unbonded and in a tightly bundled configuration and wherein the fiber filter medium further comprises a permeable outer retaining layer configured to retain the plurality of fibers in their tightly bundled configuration.

14. A shut-off valve according to claim 13 wherein the plurality of fibers consists of glass.

15. A shut-off valve according to claim 13 wherein the permeable outer retaining layer comprises one of the set consisting of woven polymeric fibers, nonwoven polymeric fibers, and a bonded polymeric fiber structure.

16. A shut-off valve according to claim 1, wherein the porous plastic material comprises at least one of the set consisting of PE, PP, polystyrene, polytetrafluoroethylene.

17. A shut-off valve according to claim 1, wherein the porous plastic material comprises a sintered ultrahigh molecular weight polyethylene.

18. A shut-off valve according to claim 1, wherein the side wall of the valve portion has a thickness in a range of 2 mm to 5 mm.

19. A shut-off valve according to claim 1, wherein the side wall of the valve portion has a thickness in a range of 3 mm to 4 mm.

20. A shut-off valve according to claim 1, wherein the moisture-reactive material comprises at least one of the set consisting of carboxymethyl-cellulose and polyacrylate.
